(19) United States
(12) Patent Application Publication
(10) Pub. No.: US 2022/0196856 A1
(43) Pub. Date: Jun. 23, 2022

(54) RADIATION DETECTOR AND RADIATION DIAGNOSTIC APPARATUS

(71) Applicant: CANON MEDICAL SYSTEMS CORPORATION, Tochigi (JP)

(72) Inventor: Go KAWATA, Moriya (JP)

(73) Assignee: CANON MEDICAL SYSTEMS CORPORATION, Tochigi (JP)

(21) Appl. No.: 17/644,866

(22) Filed: Dec. 17, 2021

(30) Foreign Application Priority Data
Dec. 17, 2020 (JP) 2020-209526

(51) Int. Cl. G01T 1/20; G01T 1/24; A61B 6/00

(52) U.S. Cl. CPC G01T 1/2018; A61B 6/037; A61B 6/4216; G01T 1/249

(57) ABSTRACT

A radiation detector according to an embodiment includes a light emitting element, an optical sensor, and a filter. The light emitting element generates light in conjunction with radiation becoming incident thereto. The optical sensor detects the light. The filter is provided between the light emitting element and the optical sensor and passes only a certain wavelength of the light so that delay time until the light is detected becomes shorter.

### CROSS-REFERENCE TO RELATED APPLICATIONS

This application is based upon and claims the benefit of priority from Japanese Patent Application No. 2020-209526, filed on Dec. 17, 2020; the entire contents of which are incorporated herein by reference.

### FIELD

Embodiments described herein relate generally to a radiation detector and a radiation diagnostic apparatus.

### BACKGROUND

Possible configurations of a radiation detector to detect radiation include a combination of a light emitting element that generates light in conjunction with radiation becoming incident thereto and an optical sensor that detects the light generated by the light emitting element. Possible examples that can be used as the optical sensor include an Avalanche Photodiode (APD) array that uses an avalanche amplification, for example.

In that situation, the light that has become incident to the optical sensor is converted into carriers through photoelectric conversion so that, after the carriers drift through the optical sensor, the avalanche amplification is carried out in a position where the electric field intensity is high, and a signal is detected. In some situations, however, variation in the position where the light becoming incident to the optical sensor is converted into the carriers through the photoelectric conversion may lead to degradation of a temporal resolution of the optical sensor.

### BRIEF DESCRIPTION OF THE DRAWINGS

FIG. 1 is a drawing for explaining a radiation detector according to an embodiment;

FIG. 2 is another drawing for explaining the radiation detector according to the embodiment;

FIG. 3 is a chart for explaining operations of an optical sensor according to the embodiment;

FIG. 4 is another chart for explaining the operations of the optical sensor according to the embodiment;

FIG. 5 is yet another chart for explaining the operations of the optical sensor according to the embodiment;

FIG. 6 is yet another chart for explaining the operations of the optical sensor according to the embodiment;

FIG. 7A is a chart for explaining operations of a filter according to the embodiment;

FIG. 7B is another chart for explaining the operations of the filter according to the embodiment;

FIG. 8 is a chart for explaining an operation of the radiation detector according to the embodiment;

FIG. 9 is a chart for explaining a filter selecting process performed by the radiation detector according to the embodiment;

FIG. 10 is another chart for explaining the filter selecting process performed by the radiation detector according to the embodiment;

FIG. 11 is a diagram for explaining an example of a radiation diagnostic apparatus according to the embodiment; and

FIG. 12 is a diagram for explaining another example of the radiation diagnostic apparatus according to the embodiment.

### DETAILED DESCRIPTION

A radiation detector provided according to an aspect of the present disclosure includes a light emitting element, an optical sensor, and a filter. The light emitting element generates light in conjunction with radiation becoming incident thereto. The optical sensor detects the light. The filter is provided between the light emitting element and the optical sensor and passes only a certain wavelength of the light so that fluctuation of delay time until the light is detected becomes smaller.

In the following sections, embodiments of a radiation detector and a radiation diagnostic apparatus will be explained in detail, with reference to the accompanying drawings.

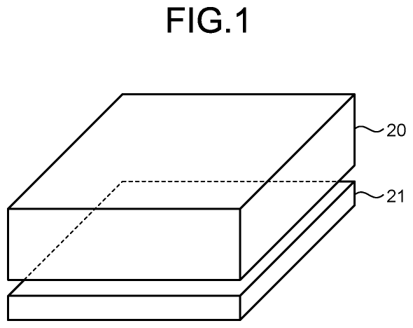

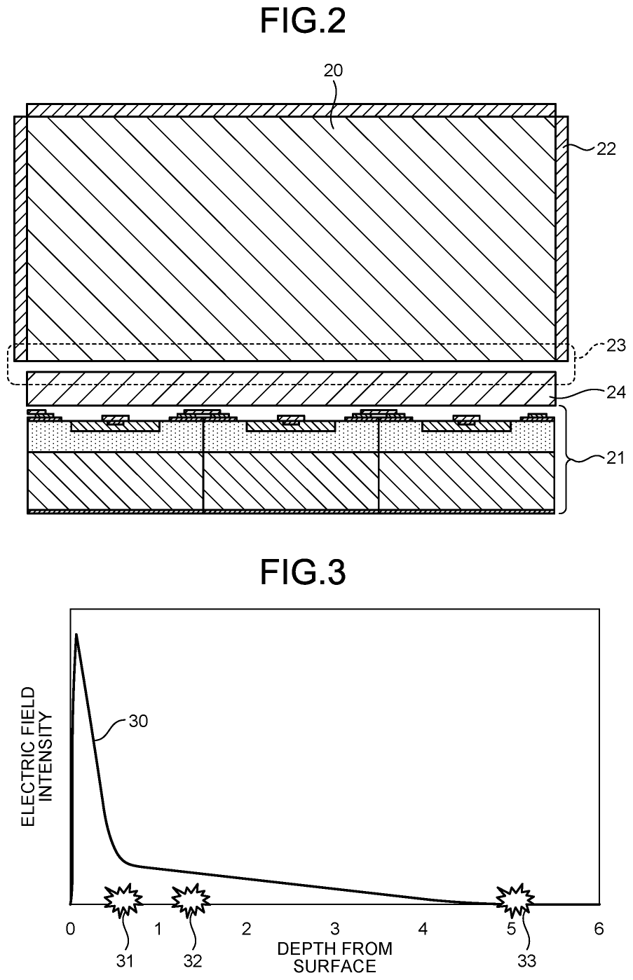

To begin with, an overall structure of a radiation detector according to an embodiment will briefly be explained, with reference to FIGS. 1 and 2. FIGS. 1 and 2 illustrate a structure of the radiation detector according to the embodiment. As illustrated in FIG. 1, the radiation detector according to the embodiment includes: a light emitting element 20 configured to generate light in conjunction with radiation becoming incident thereto; and an Avalanche Photodiode (APD) array 21 that functions as an optical sensor configured to detect the light generated by the light emitting element 20. The APD array 21 is a unit in which avalanche photodiodes configured to amplify photoelectrons through an avalanche amplification are arranged in an array formation. As the materials of the light emitting element 20 and the APD array 21, it is possible to use any of various types of materials that can be used in radiation detectors of ordinary Positron Emission Tomography (PET) apparatuses, for example.

FIG. 2 is a cross-sectional view of the structure in FIG. 1. As illustrated in FIG. 2, in the radiation detector according to the embodiment, a filter 24 configured to select only the light in a specific wavelength range is provided between the light emitting element 20 and the APD array 21. As explained later, the filter 24 is a filter configured to pass only a certain wavelength of the light so that delay time until the light generated by the light emitting element 20 is detected by the APD array 21 becomes shorter and may be configured to selectively pass blue light, for example. As the filter 24, it is possible to use a dielectric multilayer made of, for example, titanium oxide (TiO3), silicon oxide (SiO2), niobium (Nb2O5), tantalum oxide (Ta2O5), magnesium fluoride (MgF2), or the like. The filter 24 may be provided as being pasted onto the APD array 21 or may directly be formed as a film on the APD array 21. Details of the structure of the filter 24 will be explained later.

Further, on a surface of the light emitting element 20, a reflecting member 22 is further provided which is configured to reflect light having a wavelength including the wavelength passed by the filter 24. The reflecting member 22 is configured to reflect the light having a specific wavelength such as, for example, a wavelength shorter than that of green. As a result of the reflecting member 22 being provided, more photons of the light having the wavelength passed by the filter 24, i.e., of the light having the wavelength used by the APD array 21 for photon detection, become incident to the APD array 21. Detection capability of the APD array 21 is therefore improved.

In this situation, an adhesive agent 23 fills the gap between the light emitting element 20 and the APD array 21.

Next, operations performed by the APD array 21 will be explained with reference to FIGS. 3 to 6.

FIG. 3 illustrates a relationship between magnitudes of an electric field of the APD array 21 and depths from the surface, i.e., a light incident plane. In FIG. 3, the vertical axis expresses the magnitudes of the electric field, whereas the horizontal axis expresses the depths from the surface, i.e., the light incident plane. In the present example, the light incident plane of the APD array 21 denotes, for example, the plane on the top side in FIG. 2, i.e., the plane on which the filter 24 and the APD array 21 are in contact with each other. In the example in FIG. 3, electric field intensity 30 exhibits a sharp peak in a position relatively close to the surface. As for the APD array 21, in many situations, the electric field intensity exhibits a sharp peak at a specific depth, and the avalanche amplification is carried out in the peak position.

Next, an operation performed up to the time when the light that has entered the APD array 21 is detected by the APD array 21 will be explained. At first, the light that has entered through the light incident plane of the APD array 21 causes photoelectric conversion with a certain probability while advancing straight inside the device, so that carriers such as electron-hole pairs, for example, are generated. The generation of the carriers is a stochastic process. The carriers are thus distributed in various positions.

In the example illustrated in FIG. 3, for instance, by the light that has entered the APD array 21, the carriers may be formed in a position 31 in a situation, while the carriers may be formed in a position 32 in another situation, and the carriers may be formed in a position 33 in yet another situation. The distribution of the positions in which the carriers are generated by the light that has entered is dependent on wavelengths. Generally speaking, the shorter wavelength the light has, in the shallower position on average the carriers are generated. Conversely, the longer wavelength the light has, in the deeper position on average the carriers are generated.

Subsequently, due to a force received from the electric field present within the APD array 21, the carriers generated through the photoelectric conversion drift so as to move in a direction perpendicular to the light incident plane. For example, of the electron-hole pairs generated in the position 31, the position 32, and the position 33, one of the carriers advances toward the surface, whereas the other of the carriers advances in a direction away from the surface. When the carriers that have moved in this manner arrive at a location near the peak of the electric field intensity, the carriers are rapidly amplified through the avalanche amplification.

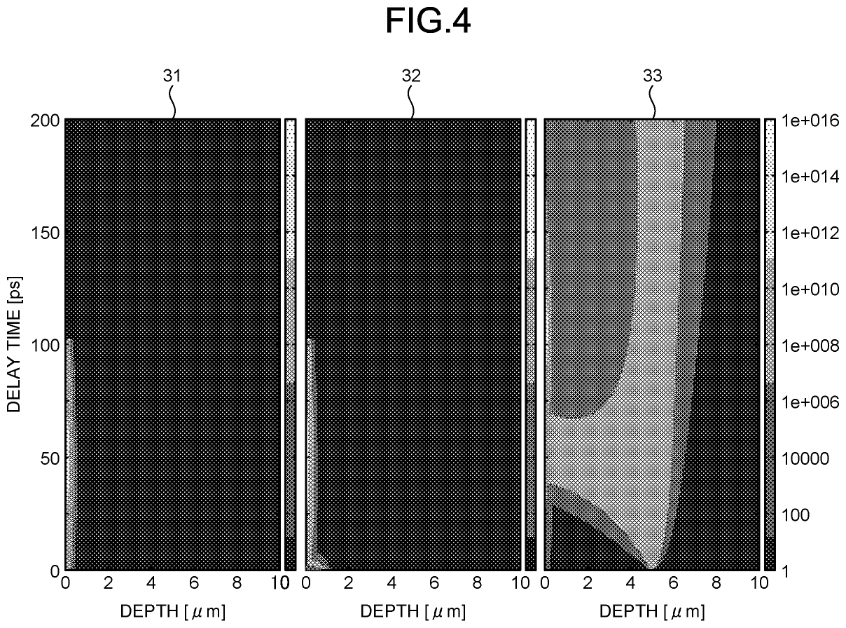

FIG. 4 illustrates the situation described above. The charts in the left, the middle, and the right sections of FIG. 4 are obtained by plotting carrier density as a mathematical function of depths from the light incident plane and delay time while the time at which the carriers are generated is expressed as 0, in correspondence with the situations where the carriers are generated in the position 31 (a shallower position), in the position 32 (a position at a medium depth), and in the position 33 (a deeper position), respectively.

As observed from the chart in the left section of FIG. 4, when the carriers are generated in the position 31, i.e., the shallower position, because the electric field intensity is high in the location where the carriers are generated, an avalanche amplification occurs instantly, so that the carrier density is instantly amplified in the vicinity of the location of the carrier generation.

Further, as observed from the chart in the middle section of FIG. 4, when the carriers are generated in the position 32, i.e., the position at the medium depth, the generated carriers drift and move toward the surface. Subsequently, an avalanche amplification occurs near the peak of the electric field intensity where the electric field intensity is high. When the carriers are generated in the position 32 at the medium depth, because it takes a little while until the generated carriers move to the vicinity of the peak of the electric field intensity, there is a time lag between the generation of the carriers and the occurrence of the avalanche amplification, in contrast to the situation where the carriers are generated in the position 31, i.e., the shallower position.

Further, as observed from the chart in the right section of FIG. 4, when the carriers are generated in the deeper position 33, the generated carriers drift and move toward the surface. Subsequently, an avalanche amplification occurs near the peak of the electric field intensity where the electric field intensity is high. However, when the carriers are generated in the position 33, because it takes a long time until the generated carriers move to the vicinity of the peak of the electric field intensity, there is a larger time lag between the generation of the carriers and the occurrence of the avalanche amplification, in contrast to the situation where the carriers are generated in the shallower position 31.

As explained herein, the more distant the position of the generation of the carriers through the photoelectric conversion is from the position of the occurrence of the avalanche amplification, the larger is the time lag between the generation of the carriers and the occurrence of the avalanche amplification, which may be a cause of degradation of the temporal resolution of the APD array 21.

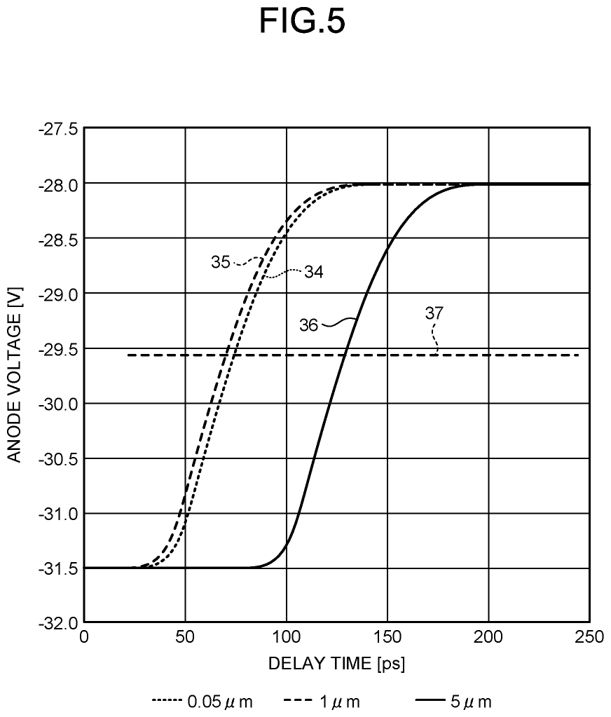

FIG. 5 presents graphs 34, 35, and 36 are graphs obtained by plotting anode voltage as a mathematical function of time, while the time at which the carriers are generated is expressed as 0, in correspondence with the situations where the carriers are generated through photoelectric conversion in the positions at the depth of 0.05 μm, 0.1 μm, and 5 μm from the surface, respectively. The threshold value 37 is a threshold value used for determining that the APD array 21 has detected photons. As observed from FIG. 5, when the depth at which the carriers are generated is deeper from the surface, there is longer delay time until anode potential reaches the threshold value, in comparison to the situation where the depth at which the carriers are generated is shallower from the surface.

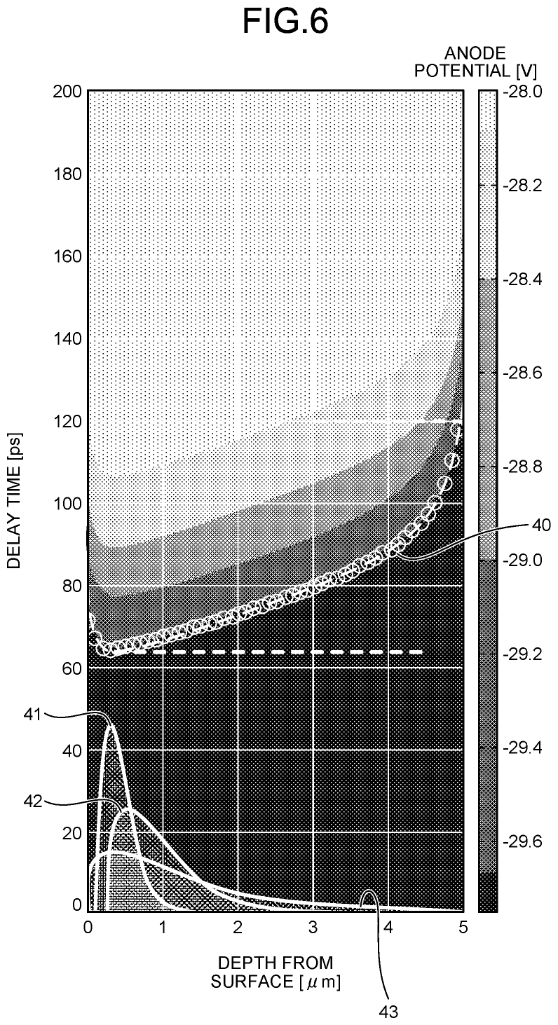

Further, FIG. 6 is a chart obtained by plotting anode potential as a mathematical function of the positions in which the carriers are generated and the delay time. Further, a graph 40 is obtained by plotting the times at which the anode potential reaches the threshold value, as a mathematical function of the positions in which the carriers are generated. As observed from the chart, the deeper the position of the generation of the carriers is, the longer is the period of time until the anode potential reaches the threshold value, i.e., the longer is the delay time until the APD array 21 detects the light. In the present example, when the carriers are generated in a deeper position on average, there is a larger variation in the delay time until the occurrence of the avalanche multiplication. As a result, temporal resolutions estimated from output signals are degraded.

Further, as explained above, the position in which the carriers are generated through the photoelectric conversion varies depending on the wavelength of the light becoming incident. A graph 41, a graph 42, and a graph 43 are obtained by plotting the probability of the occurrence of the photoelectric conversion caused by blue right, green light, and red light, respectively, as a mathematical function of depths from the surface. As observed from these graphs, the shorter the wavelength of the light is, the shallower from the surface is the position where the photoelectric conversion occurs. Conversely, the longer the wavelength of the light is, the deeper from the surface is the position where the photoelectric conversion occurs.

In view of the circumstances described above, the radiation detector according to the embodiment includes the filter 24 that is provided between the light emitting element 20 and the APD array 21 and is configured to pass only a certain wavelength of the light so that the delay time until the light generated by the light emitting element 20 is detected becomes shorter. In one example, the filter 24 may be a blue band pass filter that passes blue light.

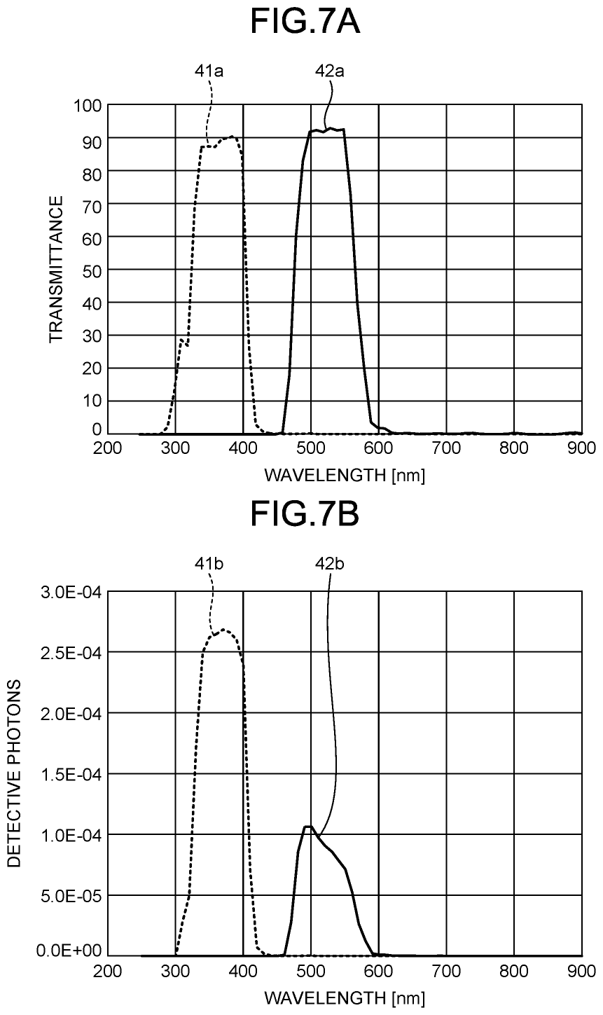

FIG. 7A illustrates transmittance of a blue band pass filter 41a and a green band pass filter 42a. The blue band pass filter 41a is a band pass filter that passes blue light and is configured to pass, for example, light having a wavelength in the range of 300 nm to 400 nm. In contrast, the green band pass filter 42a is a band pass filter that passes green light and is configured to pass, for example, light having a wavelength in the range of 450 nm to 600 nm. The blue band pass filter 41 passes light having a longer wavelength than the light passed by the green band pass filter 42a.

In FIG. 7B, a graph 41b and a graph 42b indicate intensities of the light becoming incident to the APD array 21 observed when the blue band pass filter 41a and the green band pass filter 42a are used as the filter 24, respectively. As observed from the chart, the blue band pass filter 41a is configured to selectively pass blue light, whereas the green band pass filter 42a is configured to selectively pass green light. Relative intensities between the graph 41b and the graph 42b reflect wavelength dependency of the intensity of the spectrum of the light that originally becomes incident to the APD array 21.

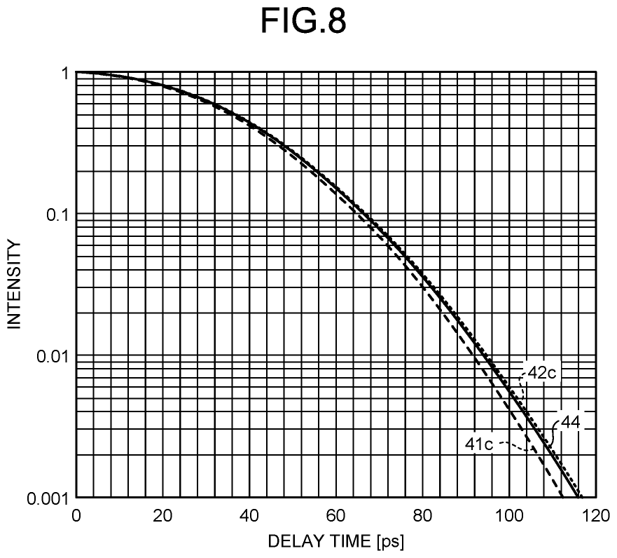

In FIG. 8, a graph 41c, a graph 42c, and a graph 44 are obtained by plotting, together with signal intensities, the delay time until the light is detected by the APD array 21, in correspondence with the situations where the blue band pass filter 41a is used as the filter 24, a green band pass filter 41b is used as the filter 24, and no filter is used, respectively. In comparison to the situations where the green band pass filter 41b is used and no filter is used, it is observed that using the blue band pulse filter 41 makes it possible to shorten the delay time, because of the elimination of the component corresponding to a green component (500 nm to 600 nm) which is a wavelength of the light having longer delay time than average delay time of the entire spectrum of the light. In other words, the radiation detector according to the embodiment is able to shorten the delay time by selecting the filter 24 capable of eliminating the wavelength of the light having the longer delay time than the average delay time of the entire spectrum of the light that becomes incident to the APD array 21.

In this situation, as explained above, it is considered that the delay time is shorter, when the distance is shorter between the position in which the carriers are generated through the photoelectric conversion and the peak position of the electric field intensity where the avalanche amplification occurs. Accordingly, the filter selected as the filter 24 may be selected so that, for example, the wavelength of the light to be passed is determined on the basis of the peak position of the electric field intensity. For example, the filter 24 may have a filter wavelength that is selected so that an average position in which the carriers are generated by the light having the wavelength is equal to the peak position of the electric field intensity.

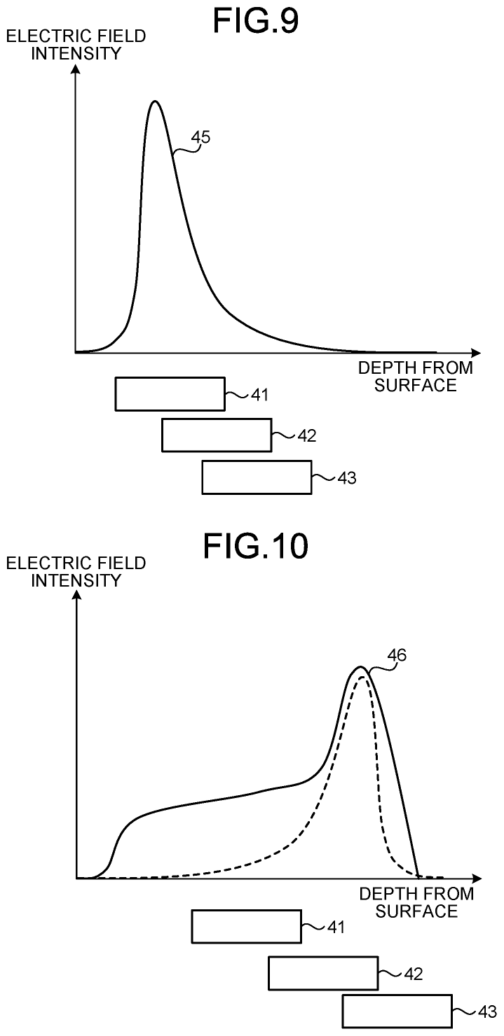

The example described above is illustrated in FIGS. 9 and 10. FIG. 9 is a chart that indicates a distribution of electric field intensity 45 in a situation where the peak position of the electric field intensity 45 is in a position relatively shallow from the surface, so as to be superimposed over distributions of the carriers generated by filters, namely, a blue band pass filter 41, a green band pass filter 42, and a red band pass filter 43. In this situation, the peak position of the electric field intensity 45 best matches the average position in which the carriers are generated while the blue band pass filter 41 is used as the filter 24, as compared among the average position in which the carriers are generated while the blue band pass filter 41 is used as the filter 24, the average position in which the carriers are generated while the green band pass filter 42 is used as the filter 24, and the average position in which the carriers are generated while the red band pass filter 43 is used as the filter 24. Accordingly, by using the blue band pass filter 41, it is possible to make the delay time the shortest.

As illustrated in FIG. 9, when the peak position of the electric field intensity 45 is in a shallow position from the surface, it is possible to shorten the delay time by selecting the filter 24 so that the average position in which the carriers are generated is closer to the incident plane than the average position when no filter is provided.

In contrast, FIG. 10 is a chart that indicates a distribution of electric field intensity 46 in a situation where the peak position of the electric field intensity 46 is in a position relatively deep from the surface, so as to be superimposed over distributions of the carriers generated by the filters, namely, the blue band pass filter 41, the green band pass filter 42, and the red band pass filter 43. In this situation, the peak position of the electric field intensity 46 best matches the average position in which the carriers are generated while the green band pass filter 42 is used as the filter 24, as compared among the average position in which the carriers are generated while the blue band pass filter 41 is used as the filter 24, the average position in which the carriers are generated while the green band pass filter 42 is used as the filter 24, and the average position in which the carriers are generated while the red band pass filter 43 is used as the filter 24. Accordingly, by using the green band pass filter 42, it is possible to make the delay time shortest. In other words, a desirable wavelength to be passed by the filter from the viewpoint of shortening the delay time is determined on the basis of a relative positional relationship between the peak position of the electric field intensity of the APD array 21 and the position in which the carriers are generated from the light having the wavelength through the photoelectric conversion.

Returning to the description of the role played by the reflecting member 22, in the radiation detector according to the present embodiment, the light emitting element 20 is further provided with the reflecting member 22 configured to reflect light having the wavelength that includes the wavelength passed by the filter 24. As a result of the reflecting member 22 configured in this manner being provided, the light having the wavelength passed by the filter 24 does not easily scatter. Consequently, the intensity of the light that has the wavelength passed by the filter 24 and becomes incident to the APD array 21 increases. As a result, characteristics of the radiation detector are improved.

For example, as illustrated in FIG. 9, when the peak position of the electric field intensity 45 is in a shallow position from the surface, it is possible to shorten the delay time of the radiation detector by providing the reflecting member 22 so that the spectrum of the light becoming incident to the APD array 21 is shifted so as to have a wavelength shorter than the wavelength of the spectrum of the light generated by the light emitting element 20.

The radiation detector according to the embodiment may be used as being incorporated in a radiation diagnostic apparatus such as a PET apparatus. In an example, the radiation detector according to the embodiment may be used, not only as a radiation detector of an ordinary PET apparatus, but also as a radiation detector intended to detect Cherenkov radiation, which requires a higher temporal resolution, by taking advantage of the feature where the filter 24 improves the temporal resolution of the detector. In that situation, the light emitting element 20 is configured to generate Cherenkov radiation in conjunction with radiation becoming incident thereto. The APD array 21 is configured to detect the Cherenkov radiation. To structure the light emitting element 20, it is possible to select from among, for example, bismuth germanium oxide (BGO) and lead compounds such as lead glass (SiO₂+PbO), lead fluoride (PbF₂), and PWO (PbWO₄). The radiation detector intended for this purpose may be incorporated in a PET apparatus of a certain type configured to generate a medical image by using the Cherenkov radiation, for example.

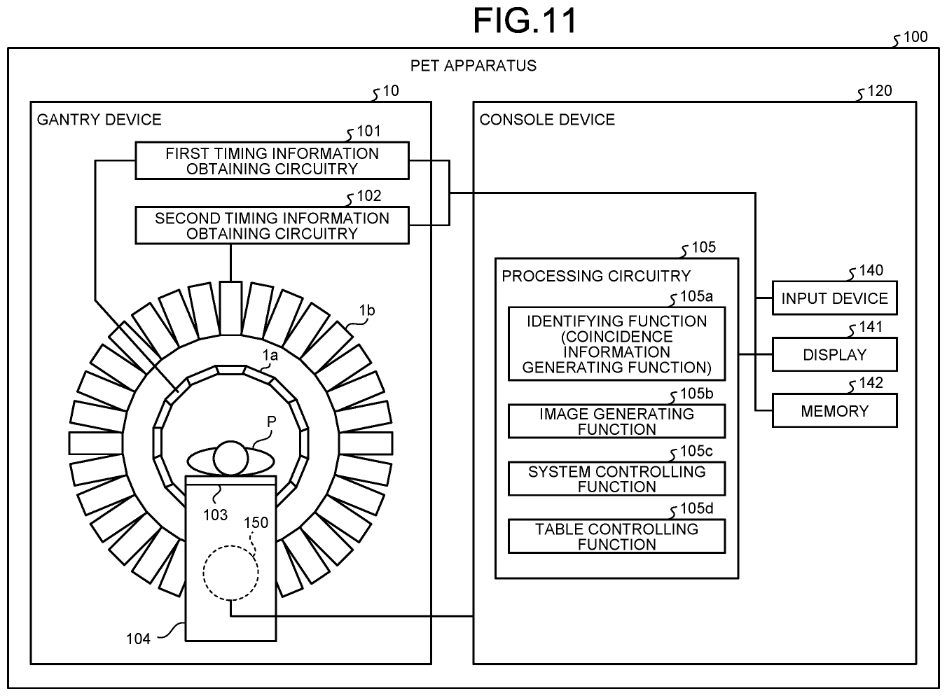

FIG. 11 illustrates an example of the abovementioned radiation diagnostic apparatus. A PET apparatus 100 illustrated in FIG. 11 is a radiation diagnostic apparatus configured to generate a medical image by using both the Cherenkov radiation and ordinary scintillation light. The PET apparatus 100 includes a gantry device 10 and a console device 120. The gantry device 10 includes: a first detector 1a configured to detect Cherenkov radiation that occurs when radiation passes; and a second detector 1b provided so as to oppose the first detector 1a while facing away from the generation source of the radiation and configured to detect energy information of the radiation. In this situation, the radiation detector according to the embodiment may be incorporated in the PET apparatus 100 as the first detector 1a.

Further, the gantry device 10 includes: first timing information obtaining circuitry 101 configured to obtain first timing information of annihilation gamma rays in the first detector 1a; and second timing information obtaining circuitry 102 configured to obtain second timing information of annihilation gamma rays in the second detector 1b, for the purpose of identifying, on the basis of the first timing information, an event of the annihilation gamma rays of which the first timing information has been obtained. The first timing information obtaining circuitry 101 and the second timing information obtaining circuitry 102 are examples of an obtaining unit.

In this situation, the PET apparatus 100 also includes a configuration of an ordinary PET apparatus. For example, the gantry device 10 includes a tabletop 103, a table 104, and a table driving unit 150. Further, the console device 120 includes processing circuitry 105, an input device 140, a display 141, and a memory 142. The processing circuitry 105 includes an identifying function 105a, an image generation function 105b, a system controlling function 105c, and a table controlling function 105d.

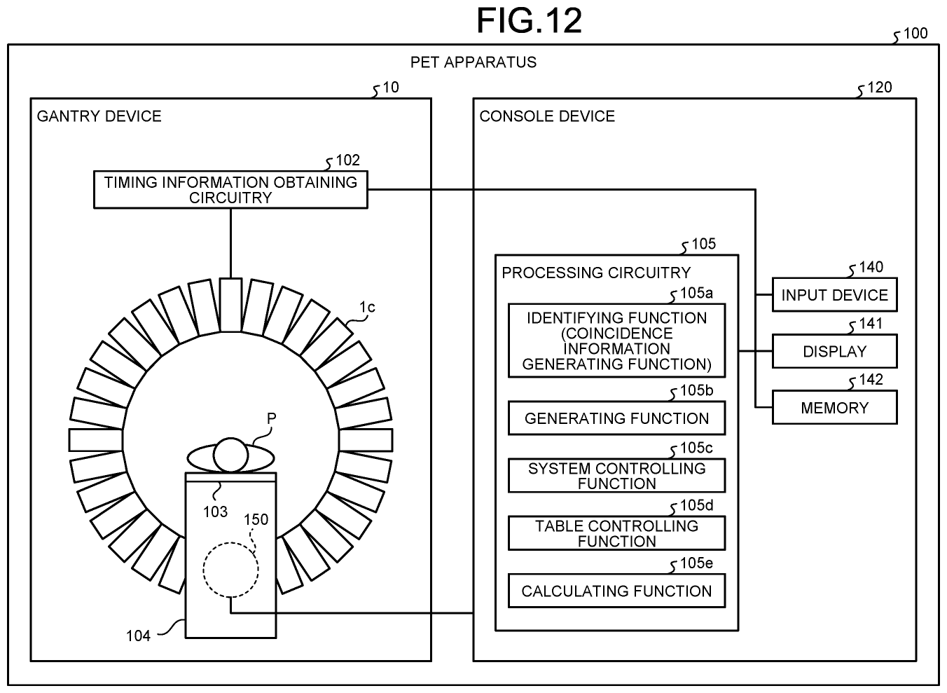

Although the radiation diagnostic apparatus of the type configured to generate a medical image by using the Cherenkov radiation was explained with reference to FIG. 11, possible embodiments are not limited to this example. For instance, as illustrated in FIG. 12, the radiation detector according to the embodiment may be incorporated in a radiation diagnostic apparatus (a PET apparatus 100) as a detector 1c of a standard PET apparatus configured to detect scintillation light. The radiation diagnostic apparatus 100 includes timing information obtaining circuitry 102 configured to obtain timing information of the radiation on the basis of data obtained by the detector 1c realized with the radiation detector according to the embodiment. The timing information obtaining circuitry 102 is an example of an obtaining unit.

According to at least one aspect of the embodiments described above, it is possible to improve the detection capabilities.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A radiation detector comprising:
a light emitting element configured to generate light in conjunction with radiation becoming incident thereto;
an optical sensor configured to detect the light; and
a filter provided between the light emitting element and the optical sensor and configured to pass only a certain wavelength of the light so that delay time until the light is detected becomes shorter.

2. The radiation detector according to claim 1, wherein the filter is selected so as to determine the wavelength of the light to be passed, based on a peak position of electric field intensity.

3. The radiation detector according to claim 2, wherein the filter is selected so that an average position in which carriers are generated by the light having the wavelength is equal to the peak position.

4. The radiation detector according to claim 1, wherein the filter is configured to eliminate a wavelength of the light having longer delay time than average delay time of an entire spectrum of the light.

5. The radiation detector according to claim 1, wherein the filter is selected so that an average position in which carriers are generated is closer to an incident plane than an average position when the filter is not provided.

6. The radiation detector according to claim 1, wherein the light emitting element is further provided with a reflecting member configured to reflect the light having a wavelength including the wavelength passed by the filter.

7. The radiation detector according to claim 1, wherein
the light emitting element is further provided with a reflecting member, and
as a result of the reflecting member being provided, a spectrum of the light becoming incident to the optical sensor is shifted so as to have a wavelength shorter than a wavelength of a spectrum of the light generated by the light emitting element.

8. The radiation detector according to claim 1, wherein
the light emitting element generates Cherenkov radiation in conjunction with the radiation becoming incident thereto, and
the optical sensor detects the Cherenkov radiation.

9. The radiation detector according to claim 1, wherein the filter is a filter configured to pass blue light.

10. A radiation diagnostic apparatus comprising:
a radiation detector that includes
a light emitting element configured to generate light in conjunction with radiation becoming incident thereto,
an optical sensor configured to detect the light; and
a filter provided between the light emitting element and the optical sensor and configured to pass only a certain wavelength of the light so that delay time until the light is detected becomes shorter; and
processing circuitry configured to obtain timing information of the radiation, based on data obtained by the radiation detector.
